(19) United States
(12) Patent Application Publication
(10) Pub. No.: US 2005/0252830 A1
(43) Pub. Date: Nov. 17, 2005

(54) Process for converting hydrocarbon condensate to fuels

(76) Inventors: Mark E. Treesh, Houston, TX (US); Randy D. Butcher, Robinson, IL (US)

Correspondence Address: Law Office of Jack E. Ebel, Suite 902, 165 South Union Boulevard, Lakewood, CO, 80228, US

(21) Appl. No.: 11/123,929

(22) Filed: May 6, 2005

Related U.S. Application Data
(60) Provisional application No. 60/570,232, filed on May 12, 2004.

(51) Int. Cl. C07C 27/06; C10G 11/08

(52) U.S. Cl. 208/115

(57) ABSTRACT

A process for treating condensate recovered from gas which is produced from a subterranean formation. Liquid condensate that is separated from gas produced from a subterranean formation is combined with a selected liquid fraction of product from a Fisher-Tropsch reactor and further processed, such as by hydrotreating, fractionating and/or hydrocracking, to produce hydrocarbon fuels or fuel blends.

### CROSS-REFERENCE TO RELATED APPLICATION

This application claims the benefit of U.S. Provisional Application No. 60/570,232 filed on May 12, 2004.

### BACKGROUND OF THE INVENTION

1. Field of the Invention

The present invention relates to a process for treating condensate recovered from gas which is produced from a subterranean formation, and more particularly, to a process wherein liquid condensate that is separated from gas produced from a subterranean formation is combined with product from a Fisher-Tropsch reactor and further processed, such as by hydrotreating, to produce hydrocarbon fuels or fuel blends.

2. Description of Related Art

Natural gas which is primarily composed of methane and other light alkanes has been discovered in large quantities throughout the world. Many of the locales in which natural gas has been discovered are far from populated regions which have significant gas pipeline infrastructure or market demand for natural gas. Due to the low density of natural gas, transportation thereof in gaseous form by pipeline or as compressed gas in vessels is expensive. Accordingly, practical and economic limits exist to the distance over which natural gas may be transported in gaseous form.

Methane found in natural gas has been used as feed to Fischer-Tropsch Gas-to-Liquids (“FT GTL”) process for the conversion of methane to heavier liquid hydrocarbons which can be further processed to fuel and fuel products. Methane is initially converted to synthesis gas consisting of carbon monoxide (CO) and hydrogen (H₂) at high temperatures (approximately 1000° C.) and high pressures (approximately 35 atmospheres). There are several types of technologies for the production of synthesis gas (CO and H₂) from methane. Among these are steam-methane reforming (SMR), partial oxidation (POX), and autothermal reforming. Synthesis gas is then fed to a Fischer-Tropsch reactor containing a catalyst, such as cobalt, ruthenium, iron, nickel or mixtures thereof, which may be present on a refractory oxide, such as aluminum, silicon or titanium oxide, that serves as a support or structural promoter. Reduction promoters, such as Pt, Ru, Pd, Re, or Cu, and activity or selectively promoters, such as K, Zr, Re, may also be employed in the catalyst as will be evident to a skilled artisan. The FT reactor is operated at an elevated temperature, for example about 200° C. to about 350° C., and pressure, for example up to about 3447 kPa, to convert carbon monoxide and hydrogen to linear and slightly branched carbon products which consist primarily of paraffins, which are predominately linear, and to a much lesser extent olefins, alcohols, aldehydes and acids. The distribution of products from an FT reactor will vary depending upon the particular FT catalyst employed as well as the operating conditions in the FT reactor. The product emanating from an FT reactor is conventionally distilled in a suitable FT fractionator into distillate fractions, which in turn are hydrotreated into suitable fuel products. Because sulfur compounds poison FT catalysts, methane is treated to remove substantially all sulfur compounds prior to generation of synthesis gas. Thus, the fuel products produced from an FT process are inherently substantially sulfur free thereby resulting in increased commercial value.

Gas produced from subterranean formations or reservoirs often has heavier hydrocarbons in varying amounts dissolved therein, depending upon the geologic conditions of deposition and upon pressure and temperature conditions in the formation or reservoir. When produced to the surface of the earth, produced gas is separated usually by means of conventional separators into natural gas and heavier hydrocarbons which are condensed into liquid at a reduced temperature and pressure. These produced liquid hydrocarbons are termed condensate and may be sour, i.e. contain sulfur compounds. Sour field condensate is currently produced and treated with a caustic solution in some field locations, which only removes the lighter sulfur compounds, i.e. methyl and ethyl mercaptans. As a result, producers are forced to transport and sell their condensate as a sour product at a reduced price. Accordingly, a need exists to upgrade such produced condensate in a cost effective and efficient manner to obtain a product having an increased market value.

### SUMMARY OF THE INVENTION

To achieve the foregoing and other objects, and in accordance with the purposes of the present invention, as embodied and broadly described herein, one characterization of the present invention is a process for converting hydrocarbon condensate. The process comprises introducing condensate recovered from a gas produced from a subterranean formation and a selected liquid fraction of a product from a Fisher-Tropsch reactor into a hydrotreater and hydrotreating the condensate and selected liquid fraction.

In another embodiment of the present invention, a process is provided for treating condensate comprising hydrotreating condensate and a Fischer-Tropsch liquid distillate in a hydrotreater

In still another embodiment of the present invention, a process is provided for treating condensate wherein a field condensate, a light Fischer-Tropsch liquid distillate and a heavy Fischer-Tropsch liquid distillate are introduced to a feed fractionator wherein a light fraction is separated from a heavy fraction. A condensate, the light fraction and hydrogen are introduced to a hydrotreater to form a hydrotreated intermediate product. The condensate is a gas plant condensate, a Fischer-Tropsch condensate or mixtures thereof. The hydrotreated intermediate product is introduced to a product fractionator wherein the hydrotreated intermediate product is separated into products.

### BRIEF DESCRIPTION OF THE DRAWINGS

The accompanying drawings, which are incorporated in and form a part of the specification, illustrate the embodiments of the present invention and, together with the description, serve to explain the principles of the invention.

In the drawings:

FIG. 1 is a block flow diagram of one embodiment of the process of the present invention;

FIG. 2 is a block flow diagram of another embodiment of the process of the present invention;

FIG. 3 is a block flow diagram of still another embodiment of the process of the present invention; and

FIG. 4 is a block flow diagram of a further embodiment of the process of the present invention.

### DETAILED DESCRIPTION OF THE PREFERRED EMBODIMENTS

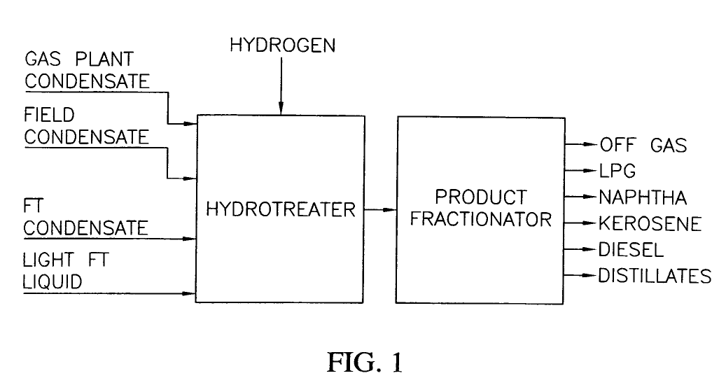

In accordance with one embodiment of the process of the present invention illustrated in FIG. 1, hydrogen, produced condensate and Fischer-Tropsch (“FT”) liquid distillate are fed to a hydrotreater wherein the condensate is desulfurized while the FT liquid distillate undergoes olefins and oxygenates saturation. The hydrotreater process and operating parameters are conventional, e.g. approximately 4137 kPa, 343° C., and 95 vol % hydrogen purity, as will be evident to a skilled artisan. The well known Fischer-Tropsch (“FT”) process involves catalytically polymerizing a synthesis gas (CO and H₂) in a suitable reactor under conditions of temperature and pressure sufficient to produced relatively long chain hydrocarbons that are suitable for further refinement into fuel products.

The product emanating from the hydrotreater, i.e. the hydrotreated intermediate product, is introduced into a product fractionator for separation into products, such as off gas, liquefied petroleum gas (“LPG”), naphtha, diesel fuel, kerosene, jet fuel, distillates, and/or waxes. It is important to note that the fuels produced by the process of the present invention, diesel fuel, kerosene and jet fuel, may be useful as fuels per se or as blend stock for fuels. As illustrated in FIG. 1, the produced condensate that is fed to the hydrotreater is a stream of field condensate and/or a stream of gas plant condensate. Field condensate is produced with gas from a subterranean formation via a well(s), has significant hydrocarbon content, is present as a liquid at wellhead conditions, and is separated from the produced gas at the wellhead or at the inlet to a gas processing plant. An exemplary field condensate generally has a C₅-C₃₀ compositional range and an end distillation point (total boiling point) of about 338° C. Gas plant condensate is liquid that has significant hydrocarbon content and is condensed from produced gas at a conventional gas processing plant. An exemplary gas plant condensate generally has a C₅-C₁₀ compositional range and an end distillation point (total boiling point) of about 165° C. The FT liquid distillate used in the embodiment of FIG. 1 is a stream of light Fischer-Tropsch (“FT”) liquid distillate and/or a stream of Fischer-Tropsch (“FT”) condensate. Light FT liquid distillate is condensed in an FT fractionator from the vapor overhead of an FT reactor by cooling the vapor to near ambient temperature. An exemplary light FT liquid distillate generally has a C₄-C₂₈ compositional range and an end distillation point (total boiling point) of about 427° C. FT condensate is an additional light liquid fraction that is further condensed from the vapor (from which the FT liquid distillate is condensed) by absorption, refrigeration or any other method evident to a skilled artisan. An exemplary FT condensate generally has a C₂-C₁₂ compositional range and an end distillation point (total boiling point) of about 200° C.

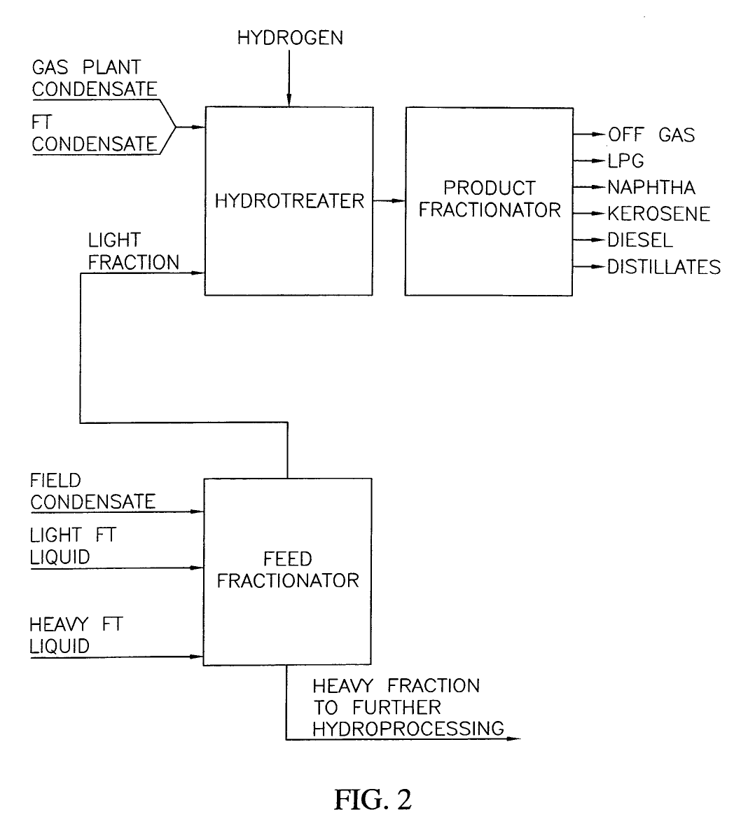

In accordance with an alternative embodiment of the process of the present invention which is illustrated in FIG. 2, the gas plant condensate and FT condensate are mixed or blended prior to introduction into a hydrotreater. The field condensate, light FT liquid distillate and heavy Fischer-Tropsch (“FT”) liquid distillate are fed to a feed fractionator with the light fraction emanating from the feed fractionator being introduced into the hydrotreater. Heavy FT liquid distillate is removed directly from the liquid present in the FT reactor. An exemplary heavy FT liquid distillate generally has a C₅-C₆₄ compositional range and an end distillation point (total boiling point) of about 638° C. The heavy fraction is removed from the feed fractionator for further processing. An exemplary light fraction generally has a C₄-C₂₀ compositional range, an end distillation point (total boiling point) of about 340° C., and is transported from the feed fractionator and introduced into the hydrotreater together with hydrogen and the combined stream of gas plant condensate and FT condensate. An exemplary heavy fraction generally has a C₁₈-C₆₄ compositional range, and an end point distillation point (total boiling point) of about 638° C. The product emanating from the hydrotreater, i.e. the hydrotreated intermediate product, is introduced into a product fractionator for separation into products, such as liquefied petroleum gas (“LPG”), naphtha, diesel fuel, kerosene, jet fuel and/or distillates.

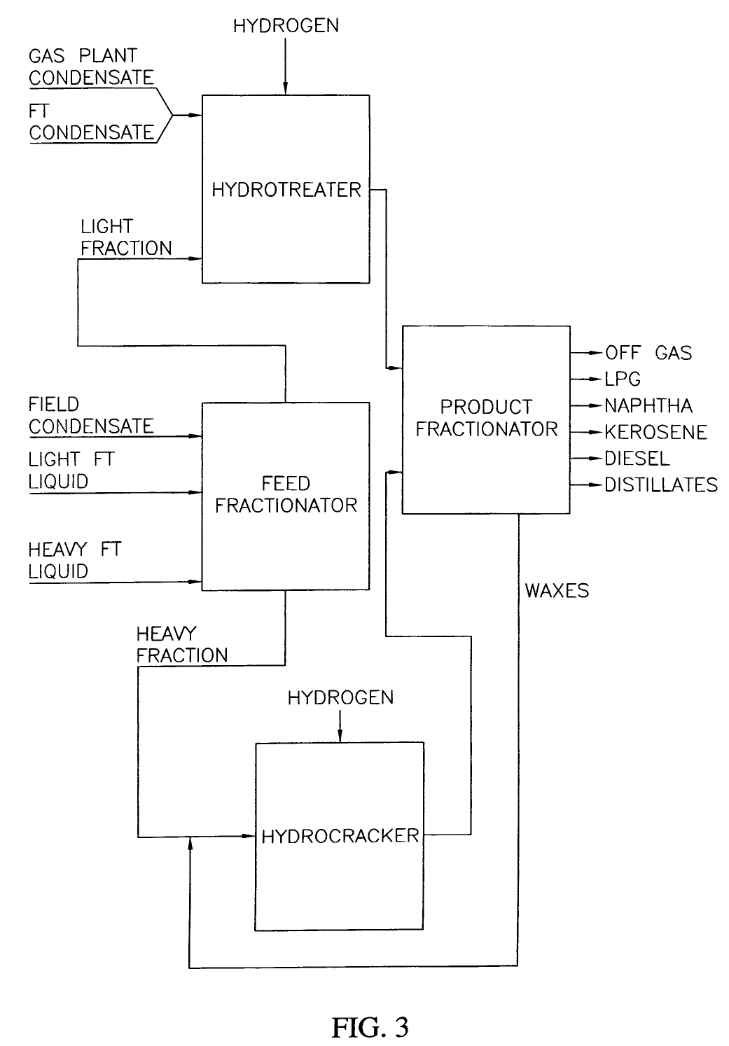

In the embodiment of the process of the present invention illustrated in FIG. 3, such further processing of the heavy fraction from the feed fractionator comprises hydrocracking wherein the heavy fraction from the feed fractionator is introduced together with waxes from the product fractionator and hydrogen into a hydrocracker. Exemplary waxes from the product fractionator generally have a C₄₆-C₆₄ compositional range, and an end distillation point (total boiling point) of about 604° C., In the hydrocracker, the heavy fraction and waxes are subject to hydrocracking, i.e. catalytically cracked or split in the presence of hydrogen to lighter carbon compounds (e.g. C₂₀+H₂→C₁₀). The effluent from the hydrocracker, i.e. the hydrocracked intermediate product, is introduced into the product fractionator for separation into product.

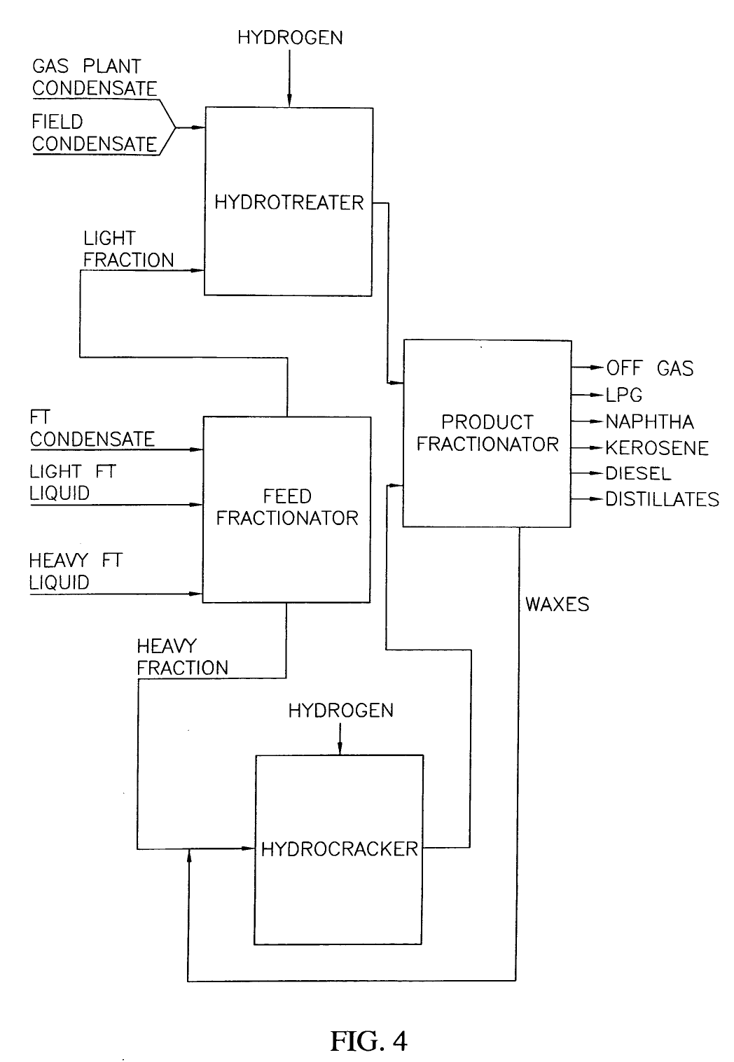

In accordance with an alternative embodiment of the process of the present invention which is illustrated in FIG. 4, the process schematic is similar to that illustrated in FIG. 3 except that field condensate instead of FT condensate is mixed or blended with gas plant condensate prior to introduction into the hydrotreater and FT condensate instead of field condensate is fed to the feed fractionator together with light FT liquid distillate and heavy FT liquid distillate. A portion of the waxes from the product fractionator in the embodiments illustrated in FIGS. 3 and 4 may be sent to a lube base oil unit (not illustrated) for production of base oils in a manner as will be evident to a skilled artisan.

The produced condensate may be condensate separated from gas produced from a given subterranean formation and/or field that is used to generate synthesis gas for the FT reactor or may be condensate separated from gas produced from a different subterranean formation and/or field. The volumetric ratio of produced condensate to FT liquid distillate that is fed to the hydrotreater in any embodiments of the present invention is, for example from about 6/5 to about 3/5, more preferably from about 4/5 to about 3/5. Preferably, the produced condensate is first stabilized in a conventional stabilizer to a Reid Vapor Pressure of about 62 kPa, more preferably 20.7 kPa, and is treated with caustic for removal of light sulfur compounds to obtain a reduced concentration, for example from about 700 to about 900 ppmw. The stabilized and treated produced condensate is then transported from the treatment site which is usually at a field location to the FT plant for introduction into the hydrotreater or feed fractionator in a manner as described above in and illustrated in FIGS. 1-3.

Hydrotreating the blend of produced condensate and an FT liquid distillate upgrades the value of the produced condensate, increases the amount of fuel product and/or fuel blend from gas produced from a subterranean formation, and still produces a fuel product and/or fuel blend having an ultra low sulfur content to meet regulatory standards.

Exemplary carbon compositional ranges and end distillation points have been set forth in the description for each of field condensate, gas plant condensate, light FT liquid distillate, FT condensate, heavy FT liquid distillate, light fraction from the feed fractionator, heavy fraction from the feed fractionator and waxes from the product fractionator. However, such exemplary compositional ranges and end distillation points are set forth merely as examples of streams suitable as feeds in the process of the present invention or produced by practice of the process of the present invention, and are not to be construed as limiting the scope of such feeds, products, and/or the process of the present invention.

While the foregoing preferred embodiments of the invention have been described and shown, it is understood that the alternatives and modifications, such as those suggested and others, may be made thereto and fall within the scope of the invention.

1. A process for converting hydrocarbon condensate comprising:
introducing condensate recovered from a gas produced from a subterranean formation, a selected liquid fraction of a product from a Fisher-Tropsch reactor, and hydrogen into a hydrotreater; and
hydrotreating said condensate and selected liquid fraction.

2. The process of claim 1 further comprising:
blending said condensate and said liquid fraction prior to introduction into said hydrotreater.

3. The process of claim 2 further comprising:
fractionating the blend of said condensate and said liquid fraction into a light fraction and a heavy fraction, wherein said step of introducing comprises introducing said light fraction into said hydrotreater.

4. The process of claim 3 further comprising:
hydrocracking said heavy fraction.

5. The process of claim 1 wherein said volumetric ratio of said condensate and said liquid fraction is from about 6/5 to about 3/5.

6. A process for treating condensate comprising:
hydrotreating condensate and a Fischer-Tropsch liquid distillate in a hydrotreater.

7. The process of claim 6 wherein said condensate and said Fischer-Tropsch liquid distillate are combined prior to introduction into said hydrotreater.

8. The process of claim 6 wherein said condensate is gas plant condensate, field condensate, or mixtures thereof.

9. The process of claim 6 wherein said Fischer-Tropsch liquid distillate is a Fischer-Tropsch condensate, a light Fischer-Tropsch liquid, or mixtures thereof.

10. The process of claim 6 further comprising:
fractionating said condensate and said Fischer-Tropsch liquid distillate that have been hydrotreated into products.

11. The process of claim 10 wherein said products are off gas, liquefied petroleum gas, naphtha, kerosene, diesel fuel, jet fuel, distillates, waxes, or mixtures thereof.

12. The process of claim 6 further comprising:
introducing a Fischer-Tropsch condensate, a light Fischer-Tropsch liquid distillate and a heavy Fischer-Tropsch liquid distillate to a feed fractionator wherein a light fraction is separated from a heavy fraction, said light fraction being said Fischer-Tropsch liquid distillate.

13. The process of claim 11 further comprising:
introducing a Fischer-Tropsch condensate, a light Fischer-Tropsch liquid distillate and a heavy Fischer-Tropsch liquid distillate to a feed fractionator wherein a light fraction is separated from a heavy fraction, said light fraction being said Fischer-Tropsch liquid distillate.

14. The process of claim 13 further comprising:
hydrocracking said heavy fraction produced in said feed fractionator.

15. The process of claim 14 further comprising:
fractionating said hydrocracked heavy portion with said hydrotreated condensate and said hydrotreated Fischer-Tropsch liquid distillate into said products.

16. The process of claim 14 further comprising:
hydrocracking said waxes with said heavy fraction produced in said feed fractionator.

17. The process of claim 16 further comprising:
fractionating said hydrocracked heavy portion and said hydrocracked waxes with said hydrotreated condensate and said hydrotreated Fischer-Tropsch liquid distillate into said products.

18. A process for treating condensate comprising:
introducing a field condensate, a light Fischer-Tropsch liquid distillate and a heavy Fischer-Tropsch liquid distillate to a feed fractionator wherein a light fraction is separated from a heavy fraction;
introducing a condensate, said light fraction and hydrogen to a hydrotreater, wherein said condensate is a gas plant condensate, a Fischer-Tropsch condensate or mixtures thereof to form a hydrotreated intermediate product; and
introducing said hydrotreated intermediate product to a product fractionator wherein said hydrotreated intermediate product is separated into products.

19. The process of claim 18 wherein said products are off gas, liquefied petroleum gas, naphtha, kerosene, diesel fuel, jet fuel, distillates, waxes, or mixtures thereof.

20. The process of claim 19 further comprising:
hydrocracking said heavy fraction produced in said feed fractionator.

21. The process of claim 20 further comprising:
introducing said hydrocracked heavy portion with said hydrotreated intermediate product into said product fractionator wherein said hydrotreated intermediate product and said hydrocracked heavy portion are separated into said products.

22. The process of claim 20 further comprising:
hydrocracking said waxes together with said heavy fraction produced in said feed fractionator to form a hydrocracked intermediate product.

23. The process of claim 22 further comprising:
introducing said hydrocracked intermediate product and said hydrotreated intermediate product into said product fractionator wherein said hydrotreated intermediate product and said hydrocracked intermediate product are separated into said products.
